(19) United States
(12) Patent Application Publication
(10) Pub. No.: US 2005/0121027 A1
(43) Pub. Date: Jun. 9, 2005

(54) Inhalable tiotropium and container therefor

(75) Inventors: Thomas Nilsson, Mariefred (SE); Mattias Myrman, Stockholm (SE); Sven Calander, Strangnas (SE); Alf Niemi, Strangnas (SE)

Correspondence Address: OBLON, SPIVAK, MCCLELLAND, MAIER & NEUSTADT, P.C., 1940 DUKE STREET, ALEXANDRIA, VA, 22314, US

(73) Assignee: Microdrug AG, Hergiswil NW (CH)

(21) Appl. No.: 10/834,037

(22) Filed: Apr. 29, 2004

(30) Foreign Application Priority Data
Dec. 3, 2003 (SE) 0303269-5
Dec. 22, 2003 (SE) 0303570-6

(51) Int. Cl. A61L 9/04; A61K 9/14; A61K 31/4745

(52) U.S. Cl. 128/200.23; 424/46; 514/291

(57) ABSTRACT

A medical product suitable for storing and delivering a pre-metered dose of tiotropium, devices containing the same, and methods of using the same.

### REFERENCE TO PRIOR APPLICATIONS

This application claims the benefit of SE 0303570-6 filed Dec. 22, 2003 and SE 0303269-5 filed Dec. 3, 2003, both of which are incorporated herein by reference.

### FIELD OF THE INVENTION

The present invention relates to a composition preferably in the form of a medical product comprising tiotropium, preferably in an inhalable pre-metered dry powder dose form, together with a finely divided excipient. The composition is preferably located/loaded in a moisture-tight, dry container. The invention further relates to a method of optimizing and preserving a fine particle dose (FPD) of a medicinal dose of a moisture sensitive tiotropium formulation during the time in-use and over the product shelf-life. The invention further provides a method for the delivery of such medical products to those in need thereof, and a method for preparing the described compositions and doses.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

Asthma and chronic obstructive pulmonary disease (COPD) affect more than 30 million people in the United States. More than 100,000 deaths each year are attributable to these conditions. Obstruction of airflow through the lungs is the characteristic feature in each of these airway diseases, and the medications utilized in treatment are often similar.

Chronic obstructive pulmonary disease (COPD) is a widespread chronic lung disorder encompassing chronic bronchitis and emphysema. The causes of COPD are not fully understood. Experience shows that the most important cause of chronic bronchitis and emphysema is cigarette smoking. Air pollution and occupational exposures may also play a role, especially when combined with cigarette smoking. Heredity also causes some emphysema cases, due to alphal anti-trypsin deficiency.

Administration of asthma drugs by an oral inhalation route is very much in focus today, because of advantages offered like rapid and predictable onset of action, cost effectiveness and high level of comfort for the user. Dry powder inhalers (DPI) and especially pre-metered DPI's are interesting as an administration tool, compared to other inhalers, because of the flexibility they offer in terms of nominal dose range, i.e. the amount of active substance that can be administered in a single inhalation.

Tiotropium, and especially the bromide salts thereof, is an effective bronchodilator. Tiotropium has a relatively fast onset and a long duration of action, which may last for 24 hours or longer. Tiotropium reduces the vagal cholinergic tone of the smooth muscle, which is the main reversible component of COPD. Tiotropium has been shown to cause quite insignificant side effects in clinical testing, dryness of mouth and constipation being perhaps the most common symptoms. Because it is often very difficult to diagnose asthma and COPD correctly and since both disorders may co-exist, it is advantageous to treat patients suffering temporary or continuous bronchial obstruction resulting in dyspnoea with a small but efficient dose of a long-acting tiotropium, preferably tiotropium bromide, because of its fast onset, long duration and small adverse side effects. Today, a bronchodilating medicament e.g. tiotropium is often co-prescribed and administered in combination with other asthma medicaments in order to provide a combined therapy, e.g. combining a bronchodilating and an anti-inflammatory treatment.

Dose efficacy depends to a great deal on delivering a stable and high fine particle dose (FPD) out of the dry powder inhaler. The FPD is the respirable dose mass out of the dry powder inhaler with an aerodynamic particle size below 5 μm. Thus, when inhaling a dose of any kind of dry medication powder it is important to obtain by mass a high fine particle fraction (FPF) of particles with an aerodynamic size preferably less than 5 μm in the inspiration air. The majority of larger particles (>5 μm) do not follow the stream of air into the many bifurcations of the airways, but get stuck in the throat and upper airways, where the medicament is not giving its intended effect, but may instead be harmful to the user. It is also important to keep the dosage to the user as exact as possible and to maintain a stable efficacy over time, and that the medicament dose does not deteriorate during normal storage. For instance, Boehringer Ingelheim KG (BI) markets tiotropium bromide under the proprietary name of Spiriva®. Surprisingly, in a recent investigation into the Spiriva® product we have found that the Spiriva®/HandiHaler® system from BI for administration by inhalation of doses contained in gelatin capsules shows poor performance and has short in-use stability.

Thus, there is a need for improvements regarding tiotropium generally, and in particular with regard to medical products comprising inhalable pre-metered dry powder doses of tiotropium, for example, with respect to achieving high and stable FPD performance from a dry powder inhaler over the in-use and lifetime of the product.

### SUMMARY OF THE INVENTION

The present invention discloses a dry composition comprising tiotropium optionally in the presence of at least one excipient and optionally with one or more further active pharmaceutical ingredients. In a preferred embodiment the composition is a medical product for use in the treatment of respiratory disorders, comprising a pre-metered dose of tiotropium in a dry powder formulation, which includes at least one finely divided excipient and optionally at least one further active pharmaceutical ingredient (API). In a further preferred embodiment the dose is directly loaded/located and sealed into a moisture-tight, dry container that provides a dry, high barrier seal against moisture.

### BRIEF DESCRIPTION OF THE DRAWINGS

The invention, together with further objects and advantages thereof, may be understood by referring to the following detailed description taken together with the accompanying drawings, in which:

FIG. 1 illustrates in a graph the results of tests S1 to S5 and HBS1 to HBS3;

FIG. 2 illustrates sorption properties of pharmaceutical excipients;

FIG. 3 illustrates in a flow-chart a method of developing a pharmaceutical composition with high FPD;

FIG. 4 illustrates in top and side views a first embodiment of a dose deposited onto a dose bed and a high barrier seal, and

FIG. 5 illustrates in top and side views a second embodiment of a dose onto a dose bed and a high barrier seal.

### DETAILED DESCRIPTION OF THE PREFERRED EMBODIMENTS

The tests described herein show that the moisture content of a gelatin capsule reduces the FPD out of the HandiHaler® by approximately 50% from the time of loading the dose into a capsule until the point in time when the product reaches the market. Loading Spiriva® (active pharmaceutical ingredient is tiotropium bromide) doses into dry containers made of materials presenting high barrier seal properties and then storing the loaded containers in 40° C. and 75% Rh, before transferring the Spiriva® doses to originator capsules and performing the same tests using HandiHaler® as before, no change can be detected in the fine particle dose (FPD), even after long periods of time. The FPD of Spiriva® in gelatin capsules, however, is further diminishing during the in-use time of the product and the FPD has been shown to drop up to another 20% after 5 days of storage in 40° C. and 75% Rh in an in-use stability test, due to the breaking of the moisture barrier of the blister package. Table 1 shows that Microdrug's C-haler, described in our U.S. Pat. No. 6,422,236 B1 incorporated herein by reference, using high barrier containers, shows a 2.6 times higher performance than HandiHaler® with respect to FPD based on metered dose.

Metered doses of the Spiriva® powder formulation are today loaded into gelatin capsules at the originator manufacturing site. A gelatin capsule contains typically 13-14% water by weight in the dose forming stage and after the capsules have been loaded they are dried in a special process in order to minimize water content. A number of dried capsules are then put in a common blister package. Details about suitable state-of-the-art capsule materials and manufacturing processes may be found in German Patent Application DE 101 26 924 A1. The remaining quantity of water in the capsule material after drying is thus enclosed in the blister package. The equilibrium between the captured air inside the package and the gelatin capsule will generate a relative humidity inside the blister package that will negatively affect the FPD of tiotropium powder out of the dry powder inhaler.

It is interesting to note that the large majority of dry powder formulations of many kinds of medicaments are not seriously affected by enclosed moisture in the capsule material or by normal storage variations in the relative humidity of the surrounding air. Examples of substances that are much more stable with respect to moisture are inhaled steroids, e.g. budesonide and fluticasone. Surprisingly, our investigation has shown tiotropium to be very much different. By some as yet unknown mechanism(s) the FPD becomes less over time when affected by very small quantities of water. Since the capsules are only used as convenient, mechanical carriers of Spiriva® doses, one solution to the moisture problem provided herein is not to use capsules at all, but rather to directly load doses into containers made of dry packaging material with high barrier seal properties during dry ambient conditions, preferably below 15% Rh.

The present invention thus provides in a highly preferred embodiment a dry, moisture-tight, directly loaded and sealed container enclosing a metered dose of tiotropium in a high FPD formulation containing at least one finely divided excipient, tiotropium powder (and/or a pharmaceutically acceptable tiotropium salt, enantiomer, racemate, hydrate, solvate, etc., including mixtures thereof, and particularly bromide) (hereinafter “tiotropium”)), the metered dose also optionally including large particles of an excipient and optionally including one or more further pharmaceutically active ingredient(s).

Another preferred embodiment of the invention is a medical product for use in the treatment of a respiratory disorder, which comprises a pre-metered dose of tiotropium in a dry powder formulation constituting at least one finely divided excipient, directly loaded and sealed into a container made so as to act as a dry high barrier seal to prevent the ingress of moisture into the powder dose. The dose is preferably further adapted for inhalation and the container is so tight that the efficacy of the dose when delivered is unaffected by moisture. In a further preferred aspect of the invention a type of inhaler is used, which may accept at least one sealed, moisture-tight container of a dose of tiotropium, to deliver a consistent and high fine particle dose over the expected shelf life of the product. In accordance with the above, the present invention also presents methods of treating respiratory diseases such as asthma and chronic obstructive pulmonary disease in individuals (or patients) in need of such treatment by administering tiotropium using the doses and/or devices and/or medical products described herein whereby the tiotropium is delivered to the pulmonary system of the individual to treat and/or alleviate the diseases being treated.

Another preferred embodiment of the present invention is a high fine particle dose (FPD) of a medical product comprising a metered dose of tiotropium, adapted for inhalation, packaged in a dry and tight container, such that the FPD when delivered is unaffected for the shelf life of the medical product by normal variations in ambient conditions during handling, storage and delivery using a DPI.

Another preferred embodiment of the present invention is a method and formulation to select suitable qualified excipients for good moisture properties and the development of a formulation to achieve high FPD out of a pre metered dry powder inhaler (DPI) both from an electrical field dosing technology and from conventional volumetric filling methods.

Another preferred embodiment of the present invention is the inclusion of one or more excipients in selected ratios together with tiotropium in a dry powder formulation, such that the actions of the excipient or excipients are to dilute the potent active ingredient and to make the flowability of the dry powder formulation acceptable for the dose forming process, and last but not least, to optimize the FPD of the metered dose.

In another aspect of the invention a type of inhaler is disclosed, which may accept at least one sealed, moisture-tight, dry container of a medical dose, for example a tiotropium dose, and deliver said dose with a consistent FPD, over the expected shelf life of the product.

In a further aspect of the invention tiotropium is mixed or formulated with one or more additional, pharmacologically active ingredient(s) and used in the treatment of respiratory disorders. The present invention further encompasses such use of tiotropium in a combined dose of medicaments in stable formulations directly loaded into a sealed, moisture-tight, dry container for insertion into a DPI, the combined dose adapted for inhalation by the user.

Further, the invention discloses a method of preventing moisturized air from a user to reach the powder in the dose prior to an inhalation and also discloses a method of making the dose available for aerosolizing in the same moment, as the seal to the container enclosing the dose is broken.

As used herein, the phrases “selected from the group consisting of,” “chosen from,” and the like include mixtures of the specified materials.

All references, patents, applications, tests, standards, documents, publications, brochures, texts, articles, instructions, etc. mentioned herein are incorporated herein by reference. Where a numerical limit or range is stated, the endpoints are included. Also, all values and subranges within a numerical limit or range are specifically included as if explicitly written out.

The term “tiotropium” as used herein is a generic term for all active forms thereof, including pharmaceutically acceptable salts, derivatives, enantiomers, racemates, hydrates, solvates or mixtures thereof. A metered dose of tiotropium normally includes one or more excipients for several purposes.

The invention container uses dry, high barrier seals impervious to moisture and other foreign matter and is adapted for insertion into a dry powder inhaler device or the container may be adapted to be a part of an inhaler device.

“Dry” as used herein means that the, e.g., walls of the container are constructed from selected materials and/or materials treated such that the walls, especially the inside wall surface of the container, cannot release water that may affect the tiotropium powder in the dose such that the FPD is reduced. As a logical consequence, container construction and materials should not be in need of processes suggested in the German publication DE 101 26 924 A 1 (US2003070679). As an example, gelatin is not a dry material and even after a special drying process gelatin still contains water. Generally, “dry” means the tiotropium FPD is not affected by the concerned material.

“High barrier seal” means a dry packaging construction or material or combinations of materials. A high barrier seal represents a high barrier against moisture, and the seal itself is ‘dry’, i.e. it cannot give off measurable amounts of water to the load of powder. A high barrier seal may for instance be made up of one or more layers of materials, i.e. technical polymers, aluminum or other metals, glass, silicon oxides etc that together constitute the high barrier seal. If the high barrier seal is a foil a 50 μm PCTFE/PVC pharmaceutical foil is a particularly useful high barrier foil especially if a two week in-use stability is desired to be achieved. For longer in-use stabilities metal foils like aluminum foils from Alcan Singen is a preferred choice.

A “high barrier container” is a mechanical construction made to harbor and enclose a dose of e.g. tiotropium. The high barrier container is built using high barrier seals constituting the walls of the container.

“Directly loaded” means that the metered dose is loaded directly into the high barrier container, i.e. without first loading the dose into e.g. a gelatin capsule, and then enclosing one or more of the primary containers (capsules) in a secondary package made of a high barrier seal material.

The high barrier containers to be loaded with tiotropium doses are preferably made out of aluminum foils approved to be in direct contact with pharmaceutical products. Aluminum foils that work properly in these aspects generally are composed of technical polymers laminated with aluminum foil to give the foil the correct mechanical properties to avoid cracking of the aluminum during forming. Sealing of the formed containers is normally done by using a thinner cover foil of pure aluminum or laminated aluminum and polymer. The container and cover foils are then sealed together using at least one of several possible methods, for instance:
using a heat sealing lacquer, through pressure and heat;
using heat and pressure to fuse the materials together;
ultrasonic welding of the materials in contact.

Tiotropium in pure form is a potent drug and it is therefore typically diluted before a dose forming step by mixing with physiologically acceptable excipients, e.g. lactose, in selected ratio(s) in order to fit a preferred method of dose forming and loading. For example, details about inhalation powders containing tiotropium in mixtures with excipients, methods of powder manufacture, use of powder and capsules for powder may be studied in the international publication WO 02/30389 A1 (U.S. Pat. No. 6,585,959 and US2002110529), Bechtold-Peters et al. Bechtold-Peters et al. describe that a physiologically acceptable excipient should be used in order to dilute the very potent tiotropium powder, such that the resulting powder mixture can be used for forming metered doses by prior art methods, well known in the industry. Bechtold-Peters et al. also disclose that in order to fill capsules consistently using prior art methods, it is important that the active compound and the excipient may be mixed easily and consistently to achieve a homogenous powder mixture. It is also important to add a suitable excipient in order to achieve good flowability of the powder mixture. Bechtold-Peters et al. show that it is advantageous to use a mixture of an excipient comprising big particles with an average size in a range 15 to 80 μm and an excipient comprising finer particles with an average size in a range 1 to 9 μm.

In the present invention, the finely divided excipient preferably comprises particles with an average size of 1 to 10 μm, including 2, 3, 4, 5, 6, 7, 8, 9 and all ranges and subranges therebetween, and optionally, but preferably, at least one additional, chemically identical or chemically different excipient comprising particles with an average size of 15 to 80 μm, including 20, 25, 30, 35, 40, 45, 50, 55, 60, 65, 70, 75 and all ranges and subranges therebetween.

In a preferred embodiment, a lower limit for volumetric dose forming is in a range 0.5 to 1 mg. Smaller doses are very difficult to produce while maintaining a low relative standard deviation between doses in the order of 10%, although the invention dose is not so limited.

Independent laboratory tests show that up to 20% of fine particles (w/w fine), i.e., a mass median aerodynamic diameter (MMAD) smaller than 10 μm, and API's, are possible to mix with larger particles, i.e., MMAD larger than 25 μm, and still maintain a stable formulation with very good FPD properties. Generally, large particles account for more than 80% (w/w) of the dose mass when using volumetric dose forming methods, although the invention is not so limited.

The present invention discloses a medicament dose comprising finely divided tiotropium mixed with at least one finely divided excipient acting as an inert diluent. If tiotropium is mixed with finely divided powder(s) of one or more additional API's, then the chosen quantity(ies) of API's may replace a part or all of the finely divided excipient as diluent, provided the added API's have suitable moisture properties, further described in the following. Different methods may be applied in formulating a dry powder tiotropium medicament, in order to make the formulation suitable for prior art filling methods. Large excipients comprising mainly large particles may or may not be made part of the tiotropium formulation at any convenient stage of the process, e.g. in order to increase flowability. Also, finely divided tiotropium may be formulated with at least one finely divided excipient and doses of such a formulation loaded into a high barrier seal container. Formulations comprising tiotropium and at least one bi-modal excipient, i.e. an excipient having a controlled fraction of fine particles as well as a fraction of large particles, is a preferred embodiment of the invention.

Independent tests show that a tiotropium formulation containing at least one finely divided excipient and developed according to methods described in this application show exceptionally good FPD data and the compositions are stable over time and during in-use time if filled into high barrier seal containers.

To develop a formulation of tiotropium with controlled moisture properties a study into the chemical and physical properties of the chosen excipient is first carried out. The sorption isotherm properties will give information with respect to how a formulation will respond to different temperatures and relative humidity in its surrounding environment. One very important question is also the “memory” of some excipients built in by the fact that it takes a very long time to reach steady state for the excipient after a disturbance in the environment. A suitable excipient for a formulation of tiotropium is an excipient like lactose monohydrate. The isotherm of lactose monohydrate has three important properties:
Low absolute water content
Low change in absolute water content after a change in relative humidity.
Highly stable in in-use temperature situations

Low absolute water content ensures that a disturbance from steady conditions will not have an impact on tiotropium when the total amount of water present in the excipient is low. The low change in absolute water content at different relative humidity ensures that the excipient has no “memory” and that it can easily be put into a steady state at a given relative humidity before filling into a high barrier container. The temperature stability ensures that adsorption and desorption inside the high barrier seal will influence the API as little as possible.

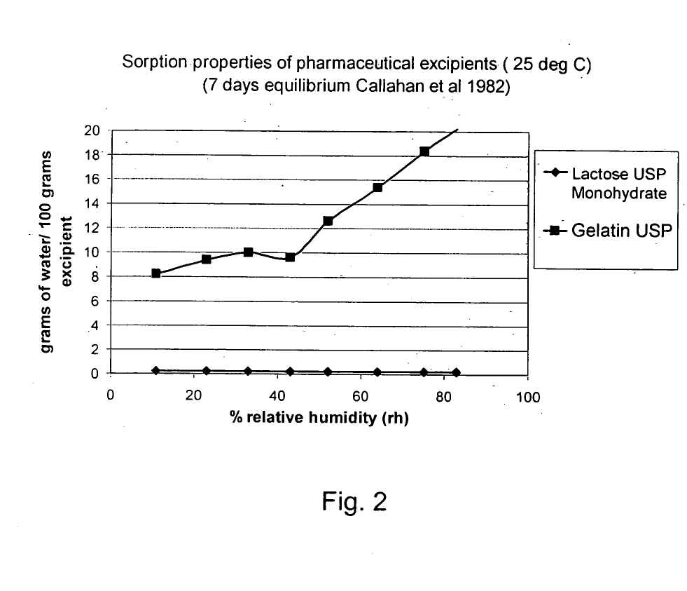

FIG. 2 shows the isotherms of gelatin today used in the Spiriva® product and lactose monohydrate as examples of a bad and a good choice of excipient or materials for a moisture sensitive tiotropium powder formulation. The effect of the excipient is normally very big when the amount of API is low. In using a volumetric dose forming method the formulation preferably possesses certain physical flow properties attained by adding larger excipient particles into the formulation. For tiotropium in the form of the Spiriva® formulation a relation between the API and the excipient or excipients is more than 1:250, which implies that a small variation in the excipient qualities, e.g. its moisture properties, may have an extremely big impact on the API and the performance of the formulation. If the electric field dosing technologies (ELFID) dose forming method is used, the relationship between API and excipient or excipients may be limited to less than 1:10 making the impact of the excipient variation much less critical than for volumetric dose forming.

A good understanding of the above-described considerations in choosing suitable excipients is necessary to ensure that the formulation of the tiotropium substance will not change in FPD if a dose of the formulation is loaded into a high barrier container, even if the container is subjected to big changes in the ambient climate. Bechtold-Peters et al. also show that suitable excipients may be found among the groups of monosaccarides, disaccarides, oligo- and polysaccarides, polyalcohols, salts or mixtures from these groups, e.g. glucose, arabinose, lactose, lactose monohydrate, lactose unhydrous, saccharose, maltose, dextrane, sorbitol, mannitol, xylitol, natriumchloride, calciumcarbonate. A preferred excipient is lactose. However, Bechtold-Peters et al. are silent regarding moisture properties of the proposed excipients. In our findings regarding the sensitivity to moisture for tiotropium the moisture properties of any proposed excipient should be investigated carefully before it is selected for inclusion in a mixture comprising tiotropium, regardless of the function of a proposed excipient. It is obvious that an excipient, which after dose forming gives off much water inside the container enclosing the dose of mixed powders may negatively affect the included active powder, such that the resulting FPD deteriorates rapidly after dose forming. Therefore, in the present invention, excipients to be mixed with tiotropium are preferably selected primarily from those excipients which have good moisture qualities in the sense that the substance will not adversely affect the active medicament FPD for the shelf life of the product regardless of normal changes in ambient conditions during storage. In this document an excipient is characterized not only by the inherent chemical formula, enantiomer etc., but also by particle size. If, e.g. lactose monohydrate is used as excipient and if the substance is present in a tiotropium formulation as a finely divided powder and as a large particle ingredient, lactose is defined as two separate excipient ingredients. Examples of suitable “dry” excipients are discussed above. In a preferred embodiment, lactose is selected as the dry excipient and more preferably, lactose monohydrate can be used in a mixture with tiotropium. Lactose as excipient has a low and constant water sorption isotherm. Excipients having a similar sorption isotherm, i.e. excipients having sorption properties not affecting a tiotropium medicament during the lifetime of the product, may also be considered for use, provided other required qualities are met.

Methods of dose forming of tiotropium include conventional mass or volumetric metering and devices and machine equipment well known to the pharmaceutical industry for, e.g., filling blister packs. WO 03027617 A1, WO 03066437 A1, WO 03066436 A1, WO 03026965 A1, WO 0244669 A1 (US2004045979) and DE 100 46 127 A1, DE 202 09 156 U1 describe examples of such volumetric and/or mass methods and devices for producing doses of medicaments in powder form. Electrostatic forming methods may also be used, for example, as disclosed in U.S. Pat. No. 6,007,630 and U.S. Pat. No. 5,699,649.

A preferred method of depositing microgram and milligram quantities of dry powders uses electric field technology (ELFID) as disclosed in U.S. Pat. No. 6,592,930 B2, the relevant disclosure of which is incorporated herein by reference. In this method powder flowability is unimportant, because powder particles are transported from a bulk source to a dose bed in a dose-forming step, not relying on the force of gravity but using primarily electric and electrostatic force technology to deposit a metered quantity of powder, i.e. a dose, onto the dose bed, which may be a blister, capsule or high barrier container as disclosed in the present invention. An advantage of this electric field dose forming process is that it is not necessary to add large excipient particles to the medicament powder, because good powder flowability is not an issue. Excipients are preferably added to the tiotropium to dilute the drug to have a pre-metered dose in the inhaler larger than 100 μg. Advantageously, the excipient is finely divided so that the mass median aerodynamic diameter (MMAD) is less than 10 μm. Tests confirm that the fine particle dose (FPD) from a dose formed by the electric field method is considerably better than the FPD from a similar dose formed by other methods common in prior art. The electric field method is also very suitable for combined doses, regardless if tiotropium is mixed with APIs or if the active medicaments are separately formed and deposited in the same container.

Ambient conditions during dose forming, loading and container sealing are preferably closely controlled. The temperature is preferably below 25° C. and relative humidity is preferably below 15% Rh. The powder formulation is preferably also kept as dry as possible during the dose forming process. Taking these precautions will limit the amount of water enclosed in the container together with the API and not enough to present a threat to the stability of the moisture sensitive substance.

In a further aspect of the invention tiotropium may be mixed or formulated with one or more other pharmacologically active ingredient(s) with an object of combining the agent with other medicament(s) to be used in a treatment of respiratory disorders. The present invention encompasses such use of tiotropium when a combination of the agent and other medicaments are deposited and sealed into a dry, moisture-tight high barrier container intended for insertion into a DPI for inhalation by the user. Examples of the additional pharmacologically active ingredients include, but are not limited to:
Inhaled steroids such as budesonid, fluticasone, rofleponide, mometasone, ciclesonide.
Anti-histamines such as epinastine, cetirizine, azelastine, fexofenadine, levocabastine, loratadine, mizolastine, ketotifene, emedastine, dirnetindene, clemastine, bamipine, cexchlorpheniramine, pheniramine, doxylamine, chlorphenoxamine, dimenhydrinate, diphenhydramine, promethazine, ebastine, desloratidine and meclozine.
Beta-mimetics such as formoterol, salmeterol, salbutamol, terbutalinsulphate.
PDE IV inhibitors: E.g. 3′,5′-cyclic nucleotide phosphodiesterases and derivates.
Adenosine A2a receptor agonists such as Ribofuranosylvanamide and derivates, substances described in publication WO 02094273 (US2003013675)

The sealed, dry, high barrier container of the invention that is directly loaded with a formulation of tiotropium may be in the form of a blister and it may, for example, comprise a flat dose bed or a formed cavity in aluminum foil or a molded cavity in a polymer material, using a high barrier seal foil against ingress of moisture, e.g. of aluminum or a combination of aluminum and polymer materials. The sealed, dry, high barrier container may form a part of an inhaler device or it may form a part of a separate item intended for insertion into an inhaler device for administration of doses. The sealed high barrier container used in the C-haler test described in the foregoing had the following data:
Container internal volume: 100 mm³
Effective diffusion area: 46 mm²
Diffusion constant: 0.044 g/m³ for 24 hours at 23° C. and differential Rh=50%

Expressed in a different way, the diffusion of water into the container was in this case at rate of 20 g/m³ per 24 hours at 23° C. at a presumed driving difference in Rh of 50%. The results from the C-haler test show that the applied container was adequate in protecting the dose for 14 days. Thus, the present invention teaches that, for example, a sealed high barrier container of the size above holding a dose of tiotropium preferably would not have a water transmission rate of more than 20 g/m³ for 24 hours at 23° C. and differential Rh=50% conditions to be suitable for an in-use time of maximum 2 weeks. The results from the C-haler test may be transposed into a set of demands put on a different type of container, e.g. a blister. A blister of similar size to the C-haler cartridge can be made using a typical high quality material like 50 μm PCTFE/PVC, which just meets the diffusion constant of the C-haler container (=0.118 g/m³ when re-calculated to at 38° C. and 90% Rh). If a device with a container of tiotropium is intended to be in use for longer periods than 2 weeks, then a more moisture tight container can be used to protect the FPD.

In US 2003/0140923 A1, a way of protecting a container filled with a dry powder is discussed using an “active approach to help if a proper high barrier seal could not be achieved”. In U.S. Pat. No. 6,130,263 and U.S. Pat. No. 5,432,214, a moisture absorbing desiccant is incorporated in the material and formed into cavities and foils to protect a product.

These applications and patents discuss the possibility of incorporating a desiccant into the material of the container or into the device or into the outer package for the device. This approach is not new and has been used for more than 20 years on the market by Turbuhaler® from AstraZeneca. Turbuhaler® has inside the device an amount of silica gel or a mixture of different types of desiccants to protect the dry powder during the in-use time and during the shelf-time. Turbuhaler® also has an outer package to protect the device during the time on the shelf before opening. Taifun® from Focus Inhalation is also using a desiccant to protect the dry powder formulation from inside the device. The amount of desiccant is normally very small in this type of construction and the demands on the high barrier seal to protect the powder remains the same if the desiccant should not be destroyed before opening of the product. In one embodiment of the invention, the medical product as described herein can also comprise one or more desiccants.

Each tiotropium formulation can be carefully checked for moisture sensitivity and a suitable protection can be selected accordingly along with consideration of the expected time in-use and the shelf life of the product.

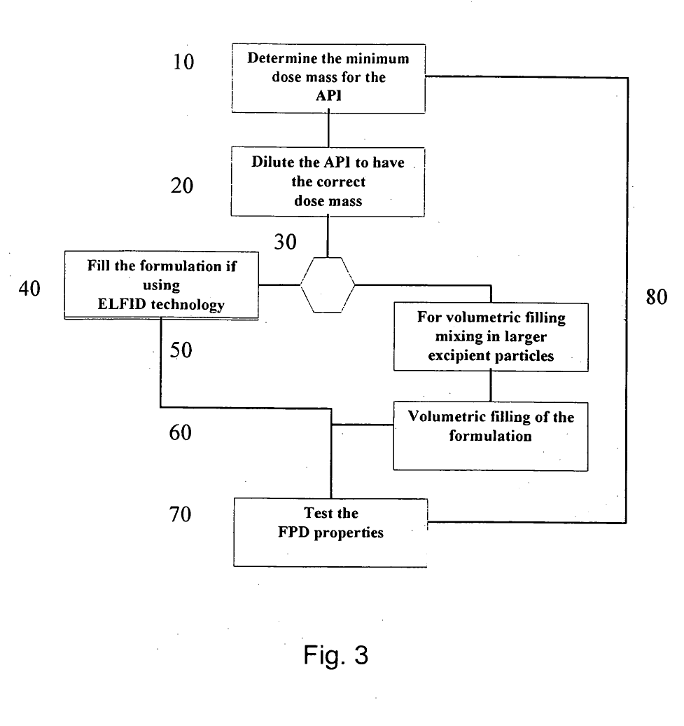

To develop a formulation of a tiotropium substance offering the best possible FPD, a method to produce an optimal formulation of the API with the excipient is preferably used. See the flow-chart illustrated in FIG. 3. Taking tiotropium, a very potent drug, a first dilution is preferably performed. The following method can be used:
Determine the minimum dose mass of the formulation for a given amount of API. Normally the minimum dose is in a range from 100 to 500 μg.
Dilute the API to have a correct minimum dose mass using an excipient having a particle size similar to the API. Preferably, this may be made by milling the API and the excipient together as a mix of powders.

If the ELFID dose forming method is to be used, then this formulation may be used directly.

If a volumetric dose forming method is to be used, a larger particle size of an excipient can be mixed into the formulation to improve physical flow properties. The mixing in of larger particles of an excipient can be made to more than 80% to get a stable powder formulation that will not segregate.

Independent tests show that large particle mixing ratios of up to more than 99.7% will not considerably diminish the FPD of the formulation for excipients of high quality.

The Dry Powder Inhaler for filling a formulation of tiotropium that is moisture sensitive makes that the inhaler device preferably meets certain criteria. In U.S. Pat. No. 5,590,645; U.S. Pat. No. 5,860,419; U.S. Pat. No. 5,873,360; U.S. Pat. No. 6,032,666; U.S. Pat. No. 6,378,519; U.S. Pat. No. 6,536,427, a pre-metered dose dry powder inhaler using peelable foils is described and some specific powders intended for inhalation mentioned. The peelable lid foils are described to be made out of a laminate comprising 50 g/m² bleach kraftpaper/12 micron polyester (PETP) fil/20 micron soft temper aluminum foil/9 g/m² vinylic peelable heat seal lacquer HSL (sealable to PVC) and a base material of a laminate comprising 100 micron PVC/45 micron soft temper aluminum foil/25 micron oriented polyamide. The heat HSL is sealed to the PVC layer of the base laminate after the powder is filled into a formed cavity in the base laminate. The process of filling is very important when powder on the heat sealable surfaces will very negatively affect the quality of the seal. Preferred filling methods do not feed the powder formulation onto the sealing surfaces during the filling process. Examples of machines that use separate machine parts to dose the powder into or onto the cavity or surface are described in WO 03027617 A1, WO 03066437 A1, WO 03066436 A1, WO 03026965 A1, WO 0244669 A1 and DE 100 46 127A1, DE20209 156U1.

A peelable HSL is typically much more sensitive and difficult to seal and as such an external high barrier package is provided to preserve the inhaler over the shelf-life and have the peelable HSL to protect the powder during the in-use time.

The above described inhaler opens the powder dose before the inhaler is ready for inhalation and is thereby exposed to the surrounding environment and the possible exhalation moist air of the user. A particularly preferred inhaler for extremely moisture sensitive drugs opens the dose during the inhalation and is insensitive to exhalation into the device.

A more secure seal with respect to moisture protection of the powder compared to the use of a peelable HSL would be to use a permanent HSL proven to withstand difficult environment conditions.

A secure high barrier seal construction of the cavities and still having the function in the device may be used to deliver tiotropium as described herein provided it can be filled with a dry excipient formulation as described in this application.

In WO 02/00280 A2 and U.S. Pat. No. 6,655,381 B2, an inhaler comprising a magazine holding a rigid unitary magazine including a plurality of integral reservoirs is described. Each reservoir will hold a pre-metered dose of dry powder sealed with a foil in an airtight manner. The foil is described as thin plastic film in WO 02/00280 A2 page 6 line 24.

The thin plastic film may be replaced with high barrier seal construction to close the reservoirs for delivery of tiotropium according to the present invention, provided such a device still functions as described herein, together with securing the moisture barrier properties, and provided it can be filled with a dry excipient formulation as described in this application.

In WO 03/66470 A1, GB 02 385 020 A, and WO 03/15857 A1 an inhaler using compartments to hold the pharmaceutical formulation is described. The compartments having a first and a second face that will be sealed with a foil and ruptured before inhalation using a sharp part inside the device. A separate part inside each compartment is designed to rapture the foil before inhalation and the documents discuss weakening special sections in the foil to make the opening easier and more reliable. This weakening of the foil could possibly be a problem to have a high barrier seal of the dose.

In WO 01/30430 A1 a dosage unit for dry powder medicaments is described. The dosage unit is possible to incorporate into a dry powder inhaler such as described in WO 02/00279, the dosage unit having a slidable chamber in a sleeve and an openable closure member possible to fit into the dry powder inhaler device. The dosage unit is described to have a cover of substantially the same diameter as the sleeve or being of a frangible material. A separate part inside the device will then push the cover open or rupture the frangible material. The present invention may be used with a dosage unit as described, provided the unit can be filled with a tiotropium composition and provided the unit comprises a high barrier seal construction for the medicament reservoirs, and if doses of tiotropium can be delivered as described herein.

In U.S. Pat. No. 2002/0033176 A1 a dry powder medicament inhalator is described, which is possible to load with a medicament cartridge. The inhalator uses an inhalation activated flow-diverting means for triggering the delivery of the medicament using a lancet to penetrate the medicament cartridge. The inhalator having a medicament cartridge as described may be used with the present invention provided the unit can be filled with a tiotropium composition and provided the cartridge comprises a high barrier seal construction for the medicament reservoirs, and if doses of tiotropium can be delivered as described herein.

Providing a device in which the film can be opened may be used provided it has a high barrier seal construction to close the reservoirs, and functions to deliver tiotropium as described herein, and provided it can be filled with a dry excipient formulation as described in this application.

To protect the FPD up to the very point of aerosolizing of the dose a method of opening the dose container a fraction of a second before the dose starts to be aerosolized is described in WO 02/24266 A1 (U.S. Pat. No. 6,651,341), the relevant disclosure of which is incorporated herein by reference. In this context it is also important to prevent a voluntary or involuntary exhalation from a user of a DPI, who is about to inhale a dose, from reaching the selected dose, because of the high moisture content in the exhalation air. In U.S. Pat. No. 6,439,227 B1, the relevant disclosure of which is incorporated herein by reference, a device is disclosed, which closes the DPI, should the user exhale, so that exhalation air does not reach the dose container and the selected dose in the DPI. The device also controls the release of a cutter and a suction nozzle such that the cutter cannot open the container and inspiration air cannot begin to aerosolize the dose until a certain selected pressure drop is present due to a suction effort by the user.

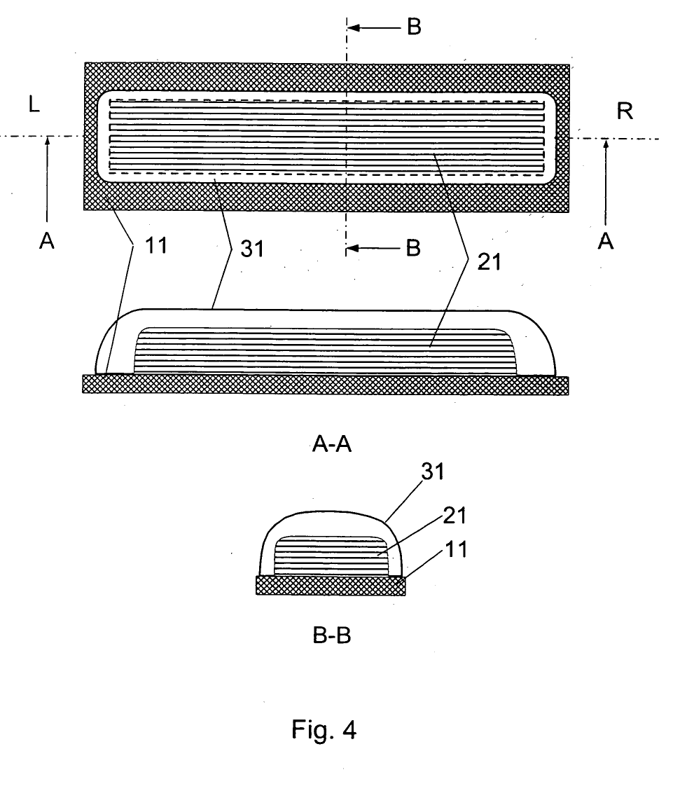

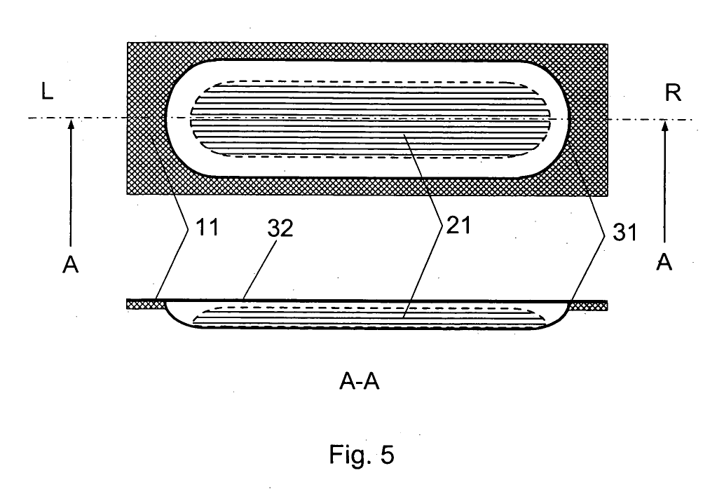

An inhaler providing a prolonged delivery of a dose during the course of a single inhalation from a high barrier seal container produced from aluminum foils constitutes a preferred embodiment of an inhaler for the delivery of the tiotropium powder formulation. An Air-razor method as described in US 2003/0192539 A1 is preferably applied in the inhaler to efficiently and gradually aerosolize the dose when delivered to the user. Surprisingly enough, applying an inhaler for a prolonged delivery and using the Air-razor method on a dose comprising tiotropium in Spiriva® formulation results in a FPD at least twice as big as that from the state-of-the-art HandiHaler® (see examples of doses illustrated in FIGS. 4 and 5). In FIGS. 4 and 5 reference numbers 11-32 of the drawings like numbers indicate like elements throughout both views of two different embodiments of doses of a dry powder medicament comprising tiotropium loaded onto a dose bed of a container as illustrated, presented here as non-limiting examples.

FIG. 4 illustrates a side and a top view of a dose 21 loaded onto a dose bed 11 of a high barrier container, the dose sealed moisture-tight by a high-barrier seal 31.

FIG. 5 illustrates a side and a top view of a dose 21 loaded onto a dose bed 11 of a high barrier container, the dose sealed moisture-tight by a high barrier seal 31 and 32.

The above written description of the invention provides a manner and process of making and using it such that any person skilled in this art is enabled to make and use the same, this enablement being provided in particular for the subject matter of the appended claims, which make up a part of the original description and including a medical product comprising a dry powder medicament dose loaded into a container for use in a dry powder inhaler, characterized in that a first component of the dry powder medicament consists of a fine particle dose of tiotropium; at least one dry excipient is present in the medicament as finely divided particles; the container constitutes a dry, high barrier seal, whereby the high barrier seal of the container prevents ingress of moisture thereby preserving the original fine particle fraction of the dry powder dose; and the dry powder medicament dose in the container is adapted for either volumetric or electric field dose forming methods.

Preferred embodiments of the invention similarly fully described and enabled include where the at least one dry excipient is presented in the medicament as finely divided particles having a diameter of 10 μm or less; and the at least one dry excipient is selected from a group of substances comprising glucose, arabinose, lactose, lactose monohydrate, lactose unhydrous, saccharose, maltose, dextrane, sorbitol, mannitol, xylitol, natriumchloride, calciumcarbonate or mixtures thereof.

Additional embodiments include where the at least one additional dry excipient is presented in the medicament as large particles having a diameter of 25 μm or more in an amount of more than 80% by weight; and the at least one dry excipient is selected from a group of substances comprising polylactides, polysaccharides, polymers, salts or mixtures thereof.

Additional embodiments include where the dry, high barrier seal is selected among the following materials, optionally in combinations: metals, including aluminum foil, thermoplastics, glass, silicon, silicon oxides.

Additional embodiments include where administration of the dry powder dose is performed by inhalation from a dry powder inhaler providing a prolonged dose delivery.

Additional embodiments include where the excipient is lactose, lactose unhydrous or lactose monohydrate.

Additional embodiments include where the dry, high barrier seal constitutes formed or flat aluminum foils, optionally laminated with polymers.

Additional embodiments include where the container forms a cavity molded from a polymer material selected to give the container high barrier seal properties.

Additional embodiments include where the container forms a cavity molded from a polymer material together with a high barrier seal providing it with high barrier seal properties.

Additional embodiments include where the container is a part of a dry powder inhaler.

Additional embodiments include where the container is a separate part adapted for insertion into a dry powder inhaler.

Additional embodiments include where the container is a separate part comprising a primary part adapted for insertion into a dry powder inhaler and a secondary part enclosing the primary part in a moisture-tight package.

Additional embodiments include where the fine particle dose of the medicament delivered from a dry powder inhaler represents more than 20% of the pre-metered dose and 40% of the delivered dose.

Additional embodiments include where the medical product is intended for use in a treatment of respiratory disorders.

Another described and enabled embodiment includes a medical combined product comprising a dry powder medicament dose loaded into a container for use in a dry powder inhaler, characterized in that a first active pharmaceutical ingredient of the dry powder medicament consists of a fine particle dose of tiotropium; an at least one dry excipient is present in the medicament as finely divided particles; the container constitutes a dry, high barrier seal, whereby the high barrier seal of the container prevents ingress of moisture thereby preserving the original fine particle fraction of the combined dose; and at least one second additional active pharmaceutical ingredient is selected from following groups of substances: inhalable steroids, nicotinamide derivatives, beta-agonists, beta-mimetics, anti-histamines, adenosine A2A receptors, PDE4 inhibitors, dopamine D2 receptor agonists.

Additional embodiments include where the at least one second additional pharmaceutical ingredient is selected from the following substances: budesonid, fluticasone, rofleponide, mometasone, ciclesonide epinastine, cetirizine, azelastine, fexofenadine, levocabastine, loratadine, mizolastine, ketotifene, emedastine, dirnetindene, clemastine, bamipine, cexchlorpheniramine, pheniramine, doxylamine, chlorphenoxamine, dimenhydrinate, diphenhydramine, promethazine, ebastine, desloratidine, meclozine, formoterol, salmeterol, salbutamol, terbutalinsulphate, 3′,5′-cyclic nucleotide phosphodiesterases and derivates, ribofuranosylvanamide and derivates.

Additional embodiments include where the at least one dry excipient is presented in the medicament as finely divided particles having a diameter of 10 μm or less, and the at least one dry excipient is selected from a group of substances comprising glucose, arabinose, lactose, lactose monohydrate, lactose unhydrous, saccharose, maltose, dextrane, sorbitol, mannitol, xylitol, natriumchloride, calciumcarbonate or mixtures thereof.

Additional embodiments include where the at least one dry excipient is presented in the medicament as large particles having a diameter of 25 μm or more in an amount of more than 80% by weight, and the at least one dry excipient is selected from a group of substances comprising polylactides, polysaccharides, polymers, salts or mixtures thereof.

Additional embodiments include where the dry, high barrier seal is selected among the following materials, optionally in combinations: metals, including aluminum foil, thermoplastics, glass, silicon, silicon oxides.

Additional embodiments include where administration of the dry powder dose is performed by inhalation from a dry powder inhaler providing a prolonged dose delivery.

Additional embodiments include where the excipient is lactose, lactose unhydrous or lactose monohydrate.

Additional embodiments include where the dry, high barrier seal constitutes formed or flat aluminum foils, optionally laminated with polymers.

Additional embodiments include where the container constitutes a cavity molded from a polymer material selected to give the container high barrier seal properties.

Additional embodiments include where the container constitutes a cavity molded from a polymer material together with a high barrier seal providing the container with high barrier seal properties.

Additional embodiments include where the container is a part of a dry powder inhaler.

Additional embodiments include where the container is a separate part adapted for insertion into a dry powder inhaler.

Additional embodiments include where the container is a separate part comprising a primary part adapted for insertion into a dry powder inhaler and a secondary part enclosing the primary part in a moisture-tight package.

Additional embodiments include where the fine particle dose of the medicament delivered from a dry powder inhaler represents more than 20% of the pre-metered dose and 40% of the delivered dose.

Additional embodiments include where the medical product is intended for use in the treatment of respiratory disorders, kits where products and inhalers are combined, methods of preparing the various compositions, doses, etc of the invention by mixing, contacting, etc (“mixing”) the required ingredients in any order, etc.

### EXAMPLES

Tiotropium is a relatively new anticholinergic agent, which is predicted to have a great potential as a bronchodilating medicament because it has a fast onset and it is long-acting, even more than 24 hours, which makes it ideal for many asthmatics. It is a potent drug and a once daily administration by inhalation is sufficient to manage asthma. If the user suffers an acute attack of asthma, then an extra administration of the tiotropitim drug brings the asthma attack under control again. However, tiotropium has problems maintaining in-use stability. This fact is documented, for example, in the report ‘COLLEGE TER BEOORDELING VAN GENEESMIDDELEN MEDICINES EVALUATION BOARD; PUBLIC ASSESSMENT REPORT; Spiriva 18 μg, inhalation powder in hard capsules; RVG 26191’ (May 21, 2002) on page 6/28 under ‘Product development and finished product’ a very short in-use stability of the Spiriva® product (9 days), a brittleness of the capsule in the blister pack ,and a very low FPD: ‘about 3 ug’ are reported. The capsules are packed in a blister made of polyvinylchloride and a protective aluminum layer. One blister card consists of two 5-cavity blisters joined along a perforated line. An aluminum peel-off foil covers the cavities. The blister allows taking one capsule at a time, so the other capsules remain protected from moist air. This polyvinylchloride film is evidently not adequate to protect SPIRIVA® capsules for more than 9 days in an in-use situation.

Details about a prior inhalation kits comprising inhalable powder of tiotropium and use of an inhaler for the administration of tiotropium may also be studied in the international publication WO 03/084502 A1 (US2003235538). Details about tiotropium compounds, medicaments based on such compounds, the use of compounds and processes for preparing compounds are described, for example, in European Patent Application 0 418 716 B1 (WO91/04252).

In the light of the above information given in the quoted report, a program was set up for testing the stabilityof the Spiriva® product according to Food and Drug Administration (FDA) recommendations.

Spiriva® is a formulation having a finely divided excipient and a larger excipient for volumetric filling into a gelatin capsule that is dried down after filling and then packaged into a tropical blister made of PVC foil. The blister is then covered with an aluminum foil. During the in-use time after opening the first capsule only the PVC foil protects the remaining 4 capsules in the blister.

A 3 week test program in accelerated conditions (40±2°/75±5 RH) for the container closure of the Spiriva® product, in this case the capsule and the blister pack, and the impact of the capsule and the blister package on the FPD was set up and tested.

Execution of Tests

Spiriva® powder formulation in bulk and Spiriva® capsules from our local pharmacy were introduced to the laboratory together with the HandiHaler® (see the following documents for a description of the HandiHaler® “Instructions for use”). The laboratory was set up to perform in-vitro tests according to European Pharmacopoeia (EP) and US Pharmacopoeia (USP) using two Andersen cascade impactors. All analytical work was then performed according to standardized methods for Physical Tests and Determinations for Aerosols, metered-dose inhalers and dry powder inhalers described in pharmacopoeias (e.g. USP 2002 <601>) using a state of the art High Performance Liquid Chromatograph (HPLC) system.

Spiriva® Tests

Test S1

Aerodynamic fine particle fraction of metered and delivered dose out of Handihaler® using Spiriva® formulation from bulk powder loaded into originator capsules during relative humidity below 10%. The test was performed with 4 kPa pressure drop over the HandiHaler® at room temperature and laboratory ambient conditions.

Test S2

Aerodynamic fine particle fraction of metered and delivered dose out of Handihaler® using commercial Spiriva® capsules purchased from our local pharmacy. Test performed with 4 kPa pressure drop over the HandiHaler® at room temperature and laboratory ambient conditions.

Test S3

An in-use stability test of the aerodynamic fine particle fraction of metered and delivered dose out of Handihaler® using commercial Spiriva® capsules purchased from our local pharmacy. From the blister holding 5 capsules one capsule was withdrawn and the remaining 4 capsules were put 4 days into 40° C. and 75% Rh. The blister containing the 4 capsules was then put in an exicator for 2 h before tests were performed. The test was performed with 4 kPa pressure drop over the HandiHaler® at room temperature and laboratory ambient conditions.

Test S4

An in-use stability test of the aerodynamic fine particle fraction of metered and delivered dose out of Handihaler® using commercial Spiriva® capsules purchased from our local pharmacy. From the blister holding 5 capsules one capsule was withdrawn and the remaining 4 capsules were put 13 days into 40° C. and 75% Rh. The blister containing the 4 capsules was then put in an exicator for 2 h before tests were performed. The test was performed with 4 kPa pressure drop over the HandiHaler® at room temperature and laboratory ambient conditions.

Test S5

An in-use stability test of the aerodynamic fine particle fraction of metered and delivered dose out of Handihaler® using commercial Spiriva® capsules purchased from our local pharmacy. From the blister holding 5 capsules one capsule was withdrawn and the remaining 4 capsules were put 21 days into 40° C. and 75% Rh. The blister containing the 4 capsules was then put in an exicator for 2 h before tests were performed. The test was performed with 4 kPa pressure drop over the HandiHaler® at room temperature and laboratory ambient conditions.

High Barrier Seal Tests

Test HBS1

An in-use stability test of the aerodynamic fine particle fraction of metered and delivered dose out of Handihaler® using Spiriva® formulation from bulk powder loaded during relative humidity below 10% into containers made to act as a high barrier seal, in this case aluminum foils from Alcan Singen Germany and then sealed to absolute tightness. The aluminum containers were put in an exicator for 2 h before the Spiriva® powder formulation was loaded from the aluminum containers into the originator capsules at a relative humidity below 10%. The test was performed with 4 kPa pressure drop over the HandiHaler® at room temperature and laboratory ambient conditions.

Test HBS2

An in-use stability test of the aerodynamic fine particle fraction of metered and delivered dose out of Handihaler® using Spiriva® formulation from bulk powder loaded during relative humidity below 10% into containers made to act as a high barrier seal, in this case aluminum foils from Alcan Singen Germany and then sealed to absolute tightness. The sealed aluminum containers were put into climate chambers for 7 days at 40° C. and 75% Rh. The aluminum containers were put in an exicator for 2 h before the Spiriva® powder formulation was loaded from the aluminum containers into the originator capsules at a relative humidity below 10%. The test was performed with 4 kPa pressure drop over the HandiHaler® at room temperature and laboratory ambient conditions.

Test HBS3

An in-use stability test of the aerodynamic fine particle fraction of metered and delivered dose out of Handihaler® using Spiriva® formulation from bulk powder loaded during relative humidity below 10% into containers made to act as a high barrier seal, in this case aluminum foils from Alcan Singen Germany and then sealed to absolute tightness. The sealed aluminum containers were put into climate chambers for 14 days at 40° C. and 75% Rh. The aluminum containers were then put in an exicator for 2 h before the Spiriva® powder formulation was loaded from the aluminum containers into the originator capsules at a relative humidity below 10%. The test was performed with 4 kPa pressure drop over the HandiHaler® at room temperature and laboratory ambient conditions.

C-haler DPI Tests

A test was also made outside the stability test program to evaluate our proprietary inhaler (see, for example, U.S. application ), the so-called C-haler, in comparison with the HandiHaler®. The C-haler cartridge used high barrier seals made out of aluminum foils from Alcan Singen Germany and the containers where filled volumetrically with 5 mg of the Spiriva® powder formulation in bulk. The test was performed using a 4 kPa pressure drop over the C-haler at room temperature and laboratory ambient conditions. The results from the Andersen impactor tests were calculated on fine particle fraction based on delivered dose as well as on metered dose and converted to FPD. The results are given in Table 1 below.

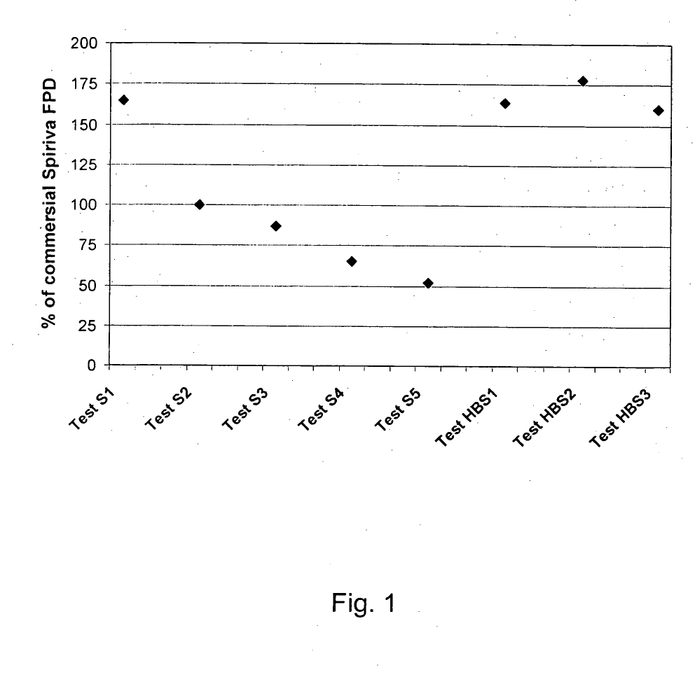

The results of tests S1-5 and HBS1-3 are plotted in FIG. 1. The Y-axis is designated ‘% of commercial Spiriva® FPD’. This relates to the FPD out from the Handihaler®, where 100% is the FPD from a fresh sample from the pharmacy.

TABLE 1
Inhaled fine particle dose (FPD) <5 μm in %

| Calculation | Spiriva ® in HandiHaler ®, | Spiriva ® in C-haler, |
|---|---|---|
| based on | commercial sample, FPD | FPD |
| Metered dose | 18% | 47% |
| Delivered dose | 36% | 56% |

Conclusion of the Tests Performed on Spiriva®

Surprisingly we have found and concluded in our tests that tiotropium is extremely sensitive to moisture and that a conventional packaging into gelatin capsules used for a majority of respiratory products will seriously affect the FPD. The results show that there is a need for a dry, moisture-tight high barrier seal enclosing the tiotropium formulation to preserve the original fine particle fraction and that gelatin is not a proper excipient or material together with the Spiriva® formulation inside a high barrier sealed container. We have also found that the tiotropium formulation can be properly protected during the in-use time if further reduction of the FPD shall be avoided.

As is clear from the above specification, a preferred embodiment of the invention is a medical product comprising a dry powder medicament dose loaded into a container for use in a dry powder inhaler, wherein the dry powder medicament dose comprises a fine particle dose of tiotropium and at least one dry excipient present in the form of finely divided particles; and wherein the container comprises a dry, high barrier seal, and the dry powder medicament dose in the container is adapted for either volumetric or electric field dose forming methods. In one preferred embodiment, the medicament dose is kept dry by the container such that, for example, the FPD is maintained at 100%, 99%, 98%, 97%, 95%, 92%, 85%, etc, for example at 40 C and 75% Rh for 5 days. Alternatively, or additionally, the sealed high barrier-comprising container of the invention preferably does not have a water transmission rate of more than 1, 3, 5, 7, 9, 11, 13, 15, 18, 20, 22, 25, 30, 35, 40, 45, etc. g/m³for 24 hours at 23° C. and differential Rh=50%.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A medical product comprising a dry powder medicament dose loaded into a container for use in a dry powder inhaler, wherein the dry powder medicament dose comprises a fine particle dose of tiotropium and at least one dry excipient present in the form of finely divided particles; and wherein the container comprises a dry, high barrier seal, and the dry powder medicament dose in the container is adapted for either volumetric or electric field dose forming methods.

2. The medical product according to claim 1, wherein the at least one dry excipient has a mass median aerodynamic diameter of 10 μm or less.

3. The medical product according to claim 2, wherein the at least one dry excipient is selected from the group consisting of glucose, arabinose, lactose, lactose monohydrate, lactose unhydrous, saccharose, maltose, dextrane, sorbitol, mannitol, xylitol, sodium chloride, calcium carbonate, and mixtures thereof.

4. The medical product according to claim 1, wherein the at least one dry excipient is selected from the group consisting of glucose, arabinose, lactose, lactose monohydrate, lactose unhydrous, saccharose, maltose, dextrane, sorbitol, mannitol, xylitol, sodium chloride, calcium carbonate, and mixtures thereof.

5. The medical product according to claim 2, comprising at least one additional dry excipient having a mass median aerodynamic diameter of 25 μm or more.

6. The medical product according to claim 1, wherein the at least one dry excipient is selected from the group consisting of polylactides, polysaccharides, polymers, salts, and mixtures thereof.

7. The medical product according to claim 1, wherein the container further comprises a desiccant.

8. The medical product according to claim 5, wherein the at least one additional dry excipient is selected from the group consisting of polylactides, polysaccharides, polymers, salts, and mixtures thereof.

9. The medical product according to claim 1, wherein the dry, high barrier seal comprises aluminum foil, and the at least one dry excipient is selected from the group consisting of lactose, lactose anhydrous, lactose monohydrate, and mixtures thereof.

10. The medical product according to claim 9, wherein the at least one dry excipient has a mass median aerodynamic diameter of 10 μm or less and further comprising at least one additional dry excipient having a mass median aerodynamic diameter of 25 μm or more.

11. The medical product according to claim 1, wherein the dry, high barrier seal is formed of at least one material selected from the group consisting of metal, thermoplastics, glass, silicon, silicon oxides, and mixtures thereof.

12. The medical product according to claim 11, wherein the dry, high barrier seal comprises aluminum foil.

13. The medical product according to claim 1, wherein the at least one dry excipient is selected from the group consisting of lactose, lactose anhydrous, lactose monohydrate, and mixtures thereof.

14. The medical product according to claim 1, wherein the dry, high barrier seal comprises a formed aluminum foil, a flat aluminum foil, or both.

15. The medical product according to claim 14, wherein the dry, high barrier seal further comprises a polymer layer laminated to the formed aluminum foil, the flat aluminum foil, or both.

16. A device comprising a dry powder inhaler and the medical product according to claim 1.

17. The device according to claim 16, wherein the device comprises a desiccant.

18. A device comprising a dry powder inhaler and the medical product according to claim 1, wherein the container of the medical product is contained within the dry powder inhaler.

19. The device according to claim 17, wherein the device comprises a desiccant.

20. A kit comprising a dry powder inhaler and the medical product according to claim 1, wherein the container is separate from the dry powder inhaler and the container is adapted for insertion into the dry powder inhaler.

21. The kit according to claim 18, wherein the device comprises a desiccant.

22. A device comprising a dry powder inhaler and the medical product according to claim 1, wherein the container is separate from the dry powder inhaler and the container comprises a first part adapted for insertion into a dry powder inhaler and a second part enclosing the first part in a moisture-tight package.

23. The medical product according to claim 1, adapted such that the fine particle dose of the medicament delivered from a dry powder inhaler represents more than 20% of the pre-metered dose and 40% of the delivered dose.

24. A method of treating asthma in an individual, comprising delivering to an individual in need thereof a fine particle dose of tiotropium through a delivery device comprising the medical product according to claim 1, such that tiotropium is delivered to the individual in an amount sufficient to treat the asthma in the individual.

25. A method of treating an obstructive pulmonary disease in an individual, comprising delivering to an individual in need thereof a fine particle dose of tiotropium through a delivery device comprising the medical product according to claim 1, such that tiotropium is delivered to the individual in an amount sufficient to treat the obstructive pulmonary disease in the individual.

26. The medical product according to claim 1, wherein the dry powder medicament further comprises at least one additional active pharmaceutical ingredient.

27. The medical product according to claim 26, wherein the high barrier container includes a desiccant.

28. The medical product according to claim 26, wherein the at least one additional active pharmaceutical ingredient is selected from the group consisting of inhalable steroids, nicotinamide derivatives, beta-agonists, beta-mimetics, anti-histamines, adenosine A2A receptors, PDE4 inhibitors, dopamine D2 receptor agonists, and mixtures thereof

29. The medical product according to claim 26, wherein the at least one additional pharmaceutical ingredient is selected from the group consisting of budesonid, fluticasone, rofleponide, mometasone, ciclesonide epinastine, cetirizine, azelastine, fexofenadine, levocabastine, loratadine, mizolastine, ketotifene, emedastine, dirnetindene, clemastine, bamipine, cexchlorpheniramine, pheniramine, doxylamine, chlorphenoxamine, dimenhydrinate, diphenhydramine, promethazine, ebastine, desloratidine, meclozine, formoterol, salmeterol, salbutamol, terbutalinsulphate, 3′,5′-cyclic nucleotide phosphodiesterases, 3′,5′-cyclic nucleotide phosphodiesterases derivatives, ribofuranosylvanamide, ribofuranosylvanamide derivatives, and mixtures thereof.

30. The medical product according to claim 26, wherein the at least one dry excipient has a mass median aerodynamic diameter of 10 μm or less.

31. The medical product according to claim 30, wherein the at least one dry excipient is selected from the group consisting of glucose, arabinose, lactose, lactose monohydrate, lactose unhydrous, saccharose, maltose, dextrane, sorbitol, mannitol, xylitol, sodium chloride, calcium carbonate, and mixtures thereof.

32. The medical product according to claim 26, wherein the at least one dry excipient is selected from a group consisting of glucose, arabinose, lactose, lactose monohydrate, lactose unhydrous, saccharose, maltose, dextrane, sorbitol, mannitol, xylitol, sodium chloride, calcium carbonate, and mixtures thereof.

33. The medical product according to claim 26, wherein the at least one dry excipient is selected from the group consisting of polylactides, polysaccharides, polymers, salts, and mixtures thereof.

34. The medical product according to claim 26, further comprising at least one additional dry excipient having a mass median aerodynamic diameter of 25 μm or more.

35. The medical product according to claim 34, wherein the at least one additional dry excipient is selected from the group consisting of polylactides, polysaccharides, polymers, salts, and mixtures thereof.

36. The medical product according to claim 26, wherein the dry, high barrier seal is formed of a material selected from the group consisting of metal, thermoplastics, glass, silicon, silicon oxides, and mixtures thereof.

37. The medical product according to claim 36, wherein the dry, high barrier seal comprises metal, which is aluminum foil.

38. The medical product according to claim 26, wherein the at least one dry excipient is selected from the group consisting of lactose, lactose anhydrous, lactose monohydrate, and mixtures thereof.

39. The medical product according to claim 26, wherein the dry, high barrier seal comprises a formed aluminum foil, a flat aluminum foil, or both.

40. The medical product according to claim 39, wherein the dry, high barrier seal further comprises a polymer layer laminated to the formed aluminum foil, the flat aluminum foil, or both.

41. A device comprising a dry powder inhaler and the medical product according to claim 26.

42. A device comprising a dry powder inhaler and the medical product according to claim 26, wherein the container of the medical product is contained within the dry powder inhaler.

43. A kit comprising a dry powder inhaler and the medical product according to claim 26, wherein the container is separate from the dry powder inhaler and the container is adapted for insertion into the dry powder inhaler.

44. A kit comprising a dry powder inhaler and the medical product according to claim 26, wherein the container is separate from the dry powder inhaler and the container comprises a first part adapted for insertion into a dry powder inhaler and a second part enclosing the first part in a moisture-tight package.

45. The medical product according to claim 26, adapted such that the fine particle dose of the medicament delivered from a dry powder inhaler represents more than 20% of the pre-metered dose and 40% of the delivered dose.

46. A method of treating asthma in an individual, comprising delivering to an individual in need thereof a fine particle dose of tiotropium through a delivery device comprising the medical product according to claim 26, such that tiotropium is delivered to the individual in an amount sufficient to treat the asthma in the individual.

47. A method of treating an obstructive pulmonary disease in an individual, comprising delivering to an individual in need thereof a fine particle dose of tiotropium through a delivery device comprising the medical product according to claim 26 such that tiotropium is delivered to the individual in an amount sufficient to treat the obstructive pulmonary disease in the individual.

48. The medical product according to claim 26, wherein the at least one additional active pharmaceutical ingredient replaces the excipient, partly or completely.
